(19) United States
(12) Patent Application Publication
(10) Pub. No.: US 2025/0176870 A1
(43) Pub. Date: Jun. 5, 2025

(54) DEVICE AND METHOD FOR DETERMINING A GLUCOSE CONCENTRATION

(71) Applicant: TRUMPF Photonic Components GmbH, Ulm (DE)

(72) Inventors: Andreas Popp, Markgröningen (DE); Berthold Schmidt, Stuttgart (DE); Holger Joachim Mönch, Vaals (NL)

(21) Appl. No.: 19/053,432

(22) Filed: Feb. 14, 2025

Related U.S. Application Data
(63) Continuation of application No. PCT/EP2023/072471, filed on Aug. 15, 2023.

(30) Foreign Application Priority Data
Aug. 19, 2022 (DE) 10 2022 121 036.7

(51) Int. Cl. A61B 5/145; A61B 5/00; A61B 5/1455

(52) U.S. Cl. CPC A61B 5/14532; A61B 5/1455; A61B 5/6803

(57) ABSTRACT

A device for determining a glucose concentration in an anterior chamber of a user's eye includes a VCSEL which emits laser light, and an optical element for influencing the laser light and/or an emergent light. The VCSEL and the optical element are configured such that the laser light enters the anterior chamber of the eye. The emergent light from the anterior chamber penetrates into the VCSEL. The device further includes an analysis unit that analyses a resulting self-mixing interference within the VCSEL to determine the glucose concentration.

### CROSS REFERENCE TO RELATED APPLICATIONS

This application is a continuation of International Application No. PCT/EP2023/072471 (WO 2024/038055 A1), filed on Aug. 15, 2023, and claims benefit to German Patent Application No. 10 2022 121 036.7, filed on Aug. 19, 2022. The aforementioned applications are hereby incorporated by reference herein.

### FIELD

Embodiments of the invention relate to a device and a method for determining a glucose concentration.

### BACKGROUND

Glucose measurement as a therapy-supporting measure was already having a major influence on the treatment of diabetes over 30 years ago. A decisive breakthrough was the availability of small, handy blood glucose meters that only required small amounts of blood as sample material and displayed the measurement result after a short amount of time. The widespread use of intensified conventional therapy (ICT) allowing insulin to be adjusted to the amount of food consumed or in the case of elevated glucose levels was one consequence of this development.

However, the biochemical reaction associated with the use of enzymatic glucose conversion was linked to the collection of blood samples (SMBG—self-monitoring of blood glucose). Even though modern devices only require drops of blood with a volume of less than 1 μl and lancing devices and lancets allow for blood collection with very little or no pain at all, the self-injury, which is necessary in each case, remains unavoidable.

Even with the availability of continuous glucose monitoring (CGM) systems, this only changed to a limited extent. These systems allow people with diabetes to obtain complete glucose histories over a period of up to 14 days. But in this case as well, at least one self-injury is necessary when inserting the glucose sensor into the subcutaneous fatty tissue. In addition, even with factory-calibrated systems that do not require invasive SMBG for calibration, such calibration is necessary to check the measured values of the CGM system or in the case of implausible glucose values.

Overall, invasive glucose measurement is painful for the person, requires time and incurs high costs for consumables.

### SUMMARY

Embodiments of the present invention provide a device for determining a glucose concentration in an anterior chamber of a user's eye. The device includes a VCSEL which emits laser light, and an optical element for influencing the laser light and/or an emergent light. The VCSEL and the optical element are configured such that the laser light enters the anterior chamber of the eye. The emergent light from the anterior chamber penetrates into the VCSEL. The device further includes an analysis unit that analyses a resulting self-mixing interference within the VCSEL to determine the glucose concentration.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a device for determining a glucose concentration in the anterior chamber

of a user's eye with a VCSEL, wherein a first optical element with a mirror surface reflects the laser light from the VCSEL, according to some embodiments;

FIG. 2 shows a further device for determining a glucose concentration in the anterior

chamber of a user's eye with a VCSEL, wherein a first optical element with a mirror surface reflects the emergent light from the chamber, according to some embodiments;

FIG. 3 shows an alternative device for determining a glucose concentration in the

anterior chamber of a user's eye with a VCSEL, wherein a first and a second optical element with respective mirror surfaces reflect the laser light and the emergent light, according to some embodiments;

FIG. 4 shows a further device for determining a glucose concentration in the anterior chamber of a user's eye with a VCSEL, wherein a third optical element with a quarter-wave plate influences the laser light from the VCSEL, wherein the reflection of the light in the anterior chamber is utilized, according to some embodiments;

FIG. 5 shows a further device for determining a glucose concentration in the anterior chamber of a user's eye with a VCSEL, wherein a fourth optical element with a liquid crystal module influences the laser light from the VCSEL, according to some embodiments;

FIG. 6 shows a further device for determining a glucose concentration in the anterior chamber of a user's eye with a VCSEL, wherein an additional reference VCSEL is provided which emits a reference laser light, according to some embodiments; and

FIG. 7 shows a glass unit with at least one of the devices of one of the preceding FIGS. 1 to 6.

### DETAILED DESCRIPTION

Embodiments of the present invention provide for a non-invasive glucose measurement.

For this purpose, it is proposed to provide a device for determining a glucose concentration in the anterior chamber of a user's eye, having a VCSEL which emits laser light and having an optical element for influencing the laser light, wherein the VCSEL (vertical-cavity surface-emitting laser) and the optical element are designed such that the laser light enters the anterior chamber of the eye, wherein emergent light from the anterior chamber of the eye penetrates into the VCSEL, and by means of an analysis unit that analyses the resulting self-mixing interference within the VCSEL the glucose concentration can be determined. Preferably, the VCSEL has a polarization grating.

The non-invasive glucose measurement is carried out by measuring the user's eye with a laser. The fluid of the anterior chamber, which consists of 98% water, contains three optically active elements (glucose, albumin, ascorbic acid). The laser beam should be coupled in here and interact with the fluid. The lower cut-off wavelength >800 nm is selected to prevent strong absorption and the unpleasant warming of the eye. The upper cut-off wavelength is <1500 nm.

The non-invasive glucose measurement takes advantage of the fact that glucose is an enantiomer and is chiral in the anterior chamber. Laser light propagating through a fluid containing glucose experiences birefringence. If linearly polarized light is now emitted, the field vector, which is aligned according to the polarization, is rotated by the chiral glucose by an angular amount that is directly proportional to the glucose concentration in the fluid in the anterior chamber. By determining the angular amount of rotation, the glucose concentration in the anterior chamber and therefore the blood glucose level can be inferred.

The laser light is coupled into the chamber of the eye and interacts with the chiral fluid. The emergent light from the chamber of the eye is then analyzed in terms of the angular amount of rotation of the polarization.

Due to the small dimensions of the anterior chamber, the interaction distance is very short and therefore requires a highly sensitive measuring system. Accordingly, a further development of the VCSEL can be equipped with a photodiode.

The emergent light fed back into the VCSEL, the polarization vector of which is rotated by the angular amount, is partially coupled back into the resonator and detected there by the photodiode. The detected signal results from the coherent interference of the laser resonator field, which represents a standing wave, and the feedback component of the emergent light. The resulting self-mixing interference exhibits a good signal-to-noise ratio, which can be analyzed to an excellent extent. This means that a glucose concentration reading can be obtained with only minor errors. The self-mixing interference and the photodiode are analyzed by a connected analysis unit.

Preferably, a polarization grating can be attached to an outcoupling facet of the VCSEL. The polarization grating polarizes the light emerging from the outcoupling facet and filters the emergent light entering the VCSEL. In this regard, the power component of the emergent light that is coupled into the resonator of the VCSEL depends on the glucose content or the angular amount of rotation in the ocular fluid as only a portion of the emergent light that is aligned with the polarization grating can return back to the VCSEL or the photodiode. The remaining portion is filtered out by the polarization grating.

Furthermore, with self-mixing interference, light of the same polarization as the light inside of the cavity can be detected, as light of a different polarization does not cause interference.

Furthermore, a VCSEL with an integrated photodiode is particularly space—and weight—saving and, compared to edge emitters, is about five times more temperature-stable with respect to the temperature dependency of the emitted wavelength.

The non-invasive glucose measurement eliminates the need for needles such that the blood glucose value can be determined without damaging a person's skin. Accordingly, the risk of infection due to contaminated needles or pain caused by needles is avoided.

As no needles are used for the non-invasive glucose measurement in contrast to an invasive glucose measurement, no consumables are required for the non-invasive glucose measurement. Accordingly, the non-invasive glucose measurement provides a method that generally offers an increased ecological and economic efficiency.

The VCSEL has an emission region on its surface from which the laser light emerges from the VCSEL.

Embodiments of the invention can also be implemented with a VCSEL array comprising a plurality of VCSELs.

Advantageously, the first optical element can have a mirror surface, wherein the mirror surface is arranged in the beam path of the emergent light such that the emergent light is reflected on the mirror surface. The laser light and the emergent light propagate along the beam path. The beam path determines the direction of the laser light or the emergent light. Through the first optical element, the emergent light can either be reflected back into the anterior chamber, where it impinges on the chiral fluid again, or at least partially past the anterior chamber along a beam path defined by the mirror surface. The optical element allows for different beam paths to be implemented.

Preferably, the anterior chamber is positioned between the VCSEL and the first optical element. In this regard, the laser light of the VCSEL enters the anterior chamber and after exiting the chamber, the laser light, which is now the emergent light from the chamber, impinges on the mirror surface of the optical element. The anterior chamber is positioned between the VCSEL and the first optical element by positioning the device in the vicinity of the user's face, for example.

In a specific further development, the laser light from the VCSEL preferably enters the anterior chamber directly and the emergent light impinges directly on the mirror surface. In this regard, the laser light from the VCSEL can be emitted into the anterior chamber without the influence of an optical element.

Alternatively or additionally, the light emerging from the VCSEL can be focused into the anterior chamber. For this purpose, an optical element in the form of a refractive and/or a diffractive lens and/or a lens made of photonic metamaterials can be used, which is arranged between the VCSEL and the chamber of the eye. Such an optical element can be attached directly to the emission region, providing a single-piece VCSEL with an optical element. This results in a high intensity in the anterior chamber and correspondingly strong interactions of the light with the chiral fluid.

Such an optical element designed as a lens can be combined with all exemplary embodiments. Here, the lens can be arranged both in the beam path of the laser light and in the beam path of the emergent light and can be combined with the other optical elements in such a way that an efficient utilization of the light from the VCSEL is ensured. In particular, the beam can be focused and/or collimated by the lens. In this regard, the beam can be focused into the anterior chamber and/or onto the emission region.

It is preferred that the anterior chamber is arranged between the first optical element and the VCSEL and that the mirror surface is preferably aligned perpendicular to the beam path of the emergent light such that the emergent light is reflected back into the anterior chamber. The emergent light reflected at the first optical element, which preferably re-enters the anterior chamber directly, exits the anterior chamber again after re-entry in order to be coupled into the VCSEL. This can lead to a further rotation of the re-entered emergent light by the angular amount such that the light emerging for the second time has an additionally rotated polarization direction.

It can be provided that the anterior chamber can be positioned between the first and a second optical element such that the beam path of the laser light passes through the anterior chamber. If the device is positioned in the vicinity of the user's face, the chamber is arranged between the first and second optical elements in such a way that the laser light from the VCSEL is first reflected at the second optical element in the direction of the anterior chamber and then penetrates into the anterior chamber. The emergent light from the chamber then impinges on the first optical element. The emergent light reflected at the first optical element does not re-enter the anterior chamber. The emergent light is guided past the anterior chamber as the first optical element is not aligned perpendicular to the beam path of the emergent light. The first optical element forms an angle with the beam path of the emergent light coming from the chamber that is different from 90° and is sufficient to guide the reflected light past the chamber. Preferably, the passing emergent light is at least indirectly coupled into the VCSEL after further reflection at the second optical element. Preferably, a lens is arranged in the emission region into which the returning light is recoupled into the VCSEL, which enables the light to be coupled in more easily and ensures that it fulfills the resonance conditions that are required for interference within the cavity of the VCSEL. For example, a refractive lens, a diffractive lens and/or a lens made of metamaterials can be used.

In particular, the beam path of the laser light from the VCSEL can pass directly to the second mirror. The laser light is emitted from the VCSEL directly onto the second optical element, where it is preferably reflected such that the beam path preferably extends directly into the anterior chamber.

In a preferred further development, the beam path of the laser light crosses at least once. For example, the beam path of the laser light from the VCSEL can cross the beam path of the emergent light. This allows for a particularly compact design of the device to be achieved as the beam paths of the emergent light and the laser light are located in the same spatial volume section.

Furthermore, the beam path of the reflected emergent light extends from the first to the second optical element. As a result, the emergent light is reflected at the first optical element and propagates in the direction of the second optical element, where it is reflected again. Preferably, it is then coupled into the VCSEL in order to effect the self-mixing interference.

A filter for the emergent light can be provided by a third optical element which produces a phase delay of a quarter wavelength when the laser light passes therethrough. In order to ensure a particularly efficient filter effect, the third optical element is positioned between the anterior chamber and the VCSEL in the beam path of the laser light from the VCSEL and the emergent light. The linearly polarized emergent light, which is rotated by the chiral fluid in the anterior chamber with respect to the laser light from the VCSEL by the angular amount, is circularly polarized by a so-called quarter-wave plate, which can constitute the third optical element. This allows the emergent light to be distinguished from scattered light based on the polarization. If the laser light from the VCSEL also passes through the quarter-wave plate and the linear polarization of the laser light is aligned in the direction of the optical axis of the quarter-wave plate, the laser light remains linearly polarized. This means that the laser light from the VCSEL and the emergent light from the anterior chamber have different polarizations and can be distinguished from one another.

Preferably, a fourth optical element can be provided, which either replaces the third optical element or is added in addition to the third optical element. The fourth optical element can be a liquid crystal module that polarizes the laser light before it enters the anterior chamber. Preferably, the polarization is rotated by an angular amount through the liquid crystal.

The liquid crystal module is controlled by a driver means such that it modulates the laser light into at least two different polarization modes, for example periodically. For example, in one modulation state, the laser light can have a polarization that coincides with the optical axis of the third optical element, and in another modulation state, the polarization direction of the laser light can be tilted by an angular amount with respect to the optical axis of the third optical element, wherein the third optical element is a quarter-wave plate. In particular, the frequency at which the different polarization modes are set is higher than the frequency of an average human eye movement such that the polarization mode is changed at least once while the eye is at rest. In crystal optics, the optical axis refers to the direction in an optically anisotropic (birefringent) crystal along which each polarization component of a light beam has the same refractive index. It is not to be confused with the optical axis of optical systems.

In a further embodiment, a reference VCSEL can emit a reference laser light, the polarization of which is selected to react with the chiral fluid in the chamber to a lesser extent than the laser light of the above-described VCSEL of the device. Preferably, the reference laser light is fully circularly polarized when it is coupled into the anterior chamber as circularly polarized light does not interact with the chiral fluid in the same way as linearly polarized light.

The reference laser light of the reference VCSEL can be collinear with the laser light of the VCSEL, wherein in particular an optical system containing an optical fiber brings the reference laser light of the reference VCSEL and the laser light of the VCSEL along a common beam path.

The device comprising the VCSEL and at least one optical element can be incorporated in a glasses unit intended to be worn by a user with a device so that a user of the glasses unit can determine his or her own blood glucose level. For example, the glasses unit can be a pair of data glasses, virtual reality glasses, augmented reality glasses or conventional eyeglasses. The device can be integrated into the glasses frame. Furthermore, a battery can be attached to the glasses unit in order to power the device.

According to the method, in order to determine a glucose concentration in the anterior chamber of a user's eye with the device, the laser light is emitted from the VCSEL, wherein the laser light enters into and then emerges from the anterior chamber, and then the emergent light is received by the VCSEL. Within the VCSEL, the light coupled into the cavity of the VCSEL produces a self-mixing interference. An analysis unit connected to the VCSEL, which preferably has a microcontroller, analyses the self-mixing interference with respect to a glucose concentration in the anterior chamber, whereby the glucose concentration is determined.

The simple and advantageous method can be used to determine the glucose concentration regularly at specific time intervals. The glucose measurement can be started after a period without measurement. The time intervals can be of the same length or individually programmed. For example, the time intervals can be selected such that the glucose measurements are taken at mealtimes, as there is then a high probability that the blood glucose level will rise for technical reasons as a result of food intake. Another alternative is to measure the blood glucose level continuously as long as the device is placed on the eye, for example by means of a pair of glasses. With each method, the temporal resolution of the measurements is different and the measurement results obtained are created in a chronological protocol of the glucose concentrations at different points in time and stored in a memory of the device. The protocol is available in digital form and can be read from the device via radio or a physical interface such as USB, for example.

The protocol can be transferred from the device to a computing device such as a smartphone or to a cloud, where it is further processed and made available to a user or a doctor, for example.

Exemplary embodiments are described below with reference to the associated drawings.

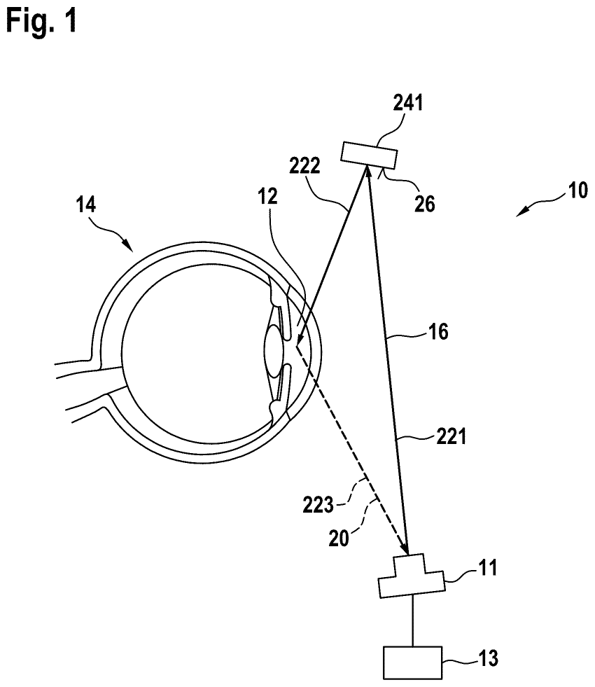

FIGS. 1 to 6 show different exemplary embodiments of a device 10 for determining a glucose concentration in the anterior chamber 12 of a user's eye 14. Each of the devices 10 utilizes a VCSEL (vertical-cavity surface-emitting laser) 11 that emits a laser light 16 that is coupled into the chamber 12.

The VCSEL 11 has an emission region on its surface from which the laser light 16 emerges from the VCSEL 11.

Preferably, the wavelength of the laser light 16 is not lower than the cut-off wavelength >800 nm and not higher than the upper cut-off wavelength <1500 nm such that a reflection of the laser light 16 in the anterior chamber 12 is possible.

The devices 10 allows for a non-invasive glucose measurement by means of a measurement using the laser light 16 on the user's eye 14. There, the fluid of the anterior chamber 12, which consists of 98% water and contains glucose, albumin and ascorbic acid, is irradiated by the laser light 16. Here, the laser light 16 interacts with the glucose in the fluid in the chamber 12, since glucose is chiral in the fluid in the chamber 12.

The field vector of the emitted and linearly polarized laser light 16 is rotated by a determined angular amount when it interacts with the chiral fluid. The laser light then exits the anterior chamber 12 as emergent light 20. The angular amount of rotation of the polarization of the emergent light 20 with respect to the polarization of the laser light 16 entering the chamber is proportional to the concentration of glucose in the fluid in the anterior chamber.

The emergent light 20 from the anterior chamber 12 penetrates into the VCSEL 11. In the cavity of the VCSEL 11, the emergent light 20 interacts with the standing wave between the Bragg mirrors of the resonator of the VCSEL 11. This results in so-called self-mixing interference between the emergent light 20 and the standing wave. The self-mixing interference can be analyzed by means of an analysis unit 13. In this regard, by analyzing the angular amount of the rotation by means of the analysis unit 13 of the device 10, the glucose concentration in the anterior chamber and therefore the blood glucose level can be inferred. Preferably, the VCSEL 11 has a polarization grating attached to the emission region from which the laser light 16 emerges and the emergent light 20 re-enters the VCSEL 11. The intensity of the self-mixing interference is determined by the polarization grating of the VCSEL 11 according to the polarization of the emergent light 20 penetrating into the VCSEL 11. The intensity can be used to determine the angular amount of polarization of the emergent light 20.

A photodiode is preferably integrated in the VCSEL 11 in order to support the detection of the coupled emergent light 20 in the VCSEL 11. The analysis unit 13 is connected to the VCSEL 11 and the photodiode.

FIG. 1 shows a device 10 with a VCSEL 11, the laser light 16 of which passes along a first beam path 221 from the VCSEL 11 to a first optical element 241. The first optical element 241 has a mirror surface 26, by means of which the emergent light 20 or the laser light 16 is influenced in such a way that it is reflected. The reflected laser light 16 passes along a second beam path 222 from the first optical element 241 to the anterior chamber 12 and enters it. In the anterior chamber 12, the laser light 16 interacts with the chiral fluid and at least part of the laser light 16 is reflected by the lens of the eye and emerges from the chamber 12 as emergent light 20. Between the first and second beam paths, the light is preferably reflected once and changes its direction of propagation.

After exiting the anterior chamber 12, the emergent light 20 is recoupled into the VCSEL 11 along a third beam path 223 that extends from the chamber 12 to the VCSEL 11.

Alternatively, the laser light 16 can first be coupled into the anterior chamber 12 before it emerges from the chamber 12 as emergent light 20 after reflection in the chamber 12 or at the iris of the eye 14 and is reflected at an optical element before re-entering the VCSEL.

In principle, the anterior chamber 12 is arranged between the first optical element 241 and the VCSEL 11, wherein this may also be the case in the following exemplary embodiments.

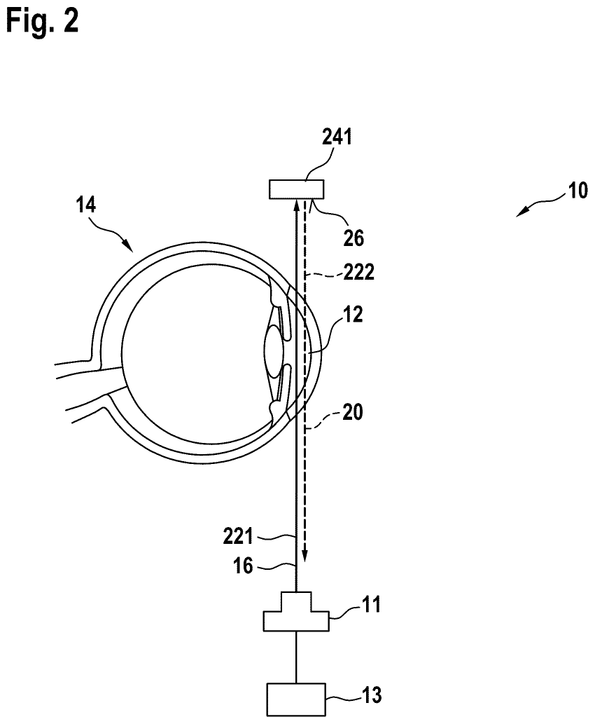

In FIG. 2, the first optical element 24, i.e., the mirror that the light first impinges upon, is arranged in the first beam path 221 after the anterior chamber 12. The mirror surface 26 is aligned perpendicular to the first beam path 221 such that the emergent light 20 is reflected back into the anterior chamber along a second beam path 222, which extends substantially parallel to the first beam path 221. This allows the polarization vector of the light to be rotated even further by an angular amount. After re-emerging from the anterior chamber 12, the emergent light 20 re-enters the VCSEL 11, wherein the emergent light 20 propagates along the second beam path 222 and thereby in the opposite direction to the laser light 16.

Alternatively or additionally, the light 16 emerging from the VCSEL 11 can be focused into the anterior chamber by means of an optical element. For this purpose, an optical element in the form of a refractive and/or a diffractive lens and/or a lens made of photonic metamaterials can be used, which is arranged between the VCSEL 16 and the chamber 12. Such an optical element can be attached directly to the emission region of the VCSEL 11, providing a single-piece VCSEL 11 with an attached optical element. This results in a high intensity in the anterior chamber 12 and correspondingly strong interactions of the light 16 with the chiral fluid.

Such an optical element designed as a lens can be combined with all exemplary embodiments. Here, the lens can be arranged both in the beam path 221, 222, 223, 224 of the laser light 16 and in the beam path 221, 222, 223, 224 of the emergent light 20 and can be combined with the other optical elements 241, 242, 243, 244 in such a way that an efficient utilization of the light from the VCSEL 11 is ensured. In particular, the beam can be focused and/or collimated by the lens. In this regard, the beam can be focused into the anterior chamber 12 and/or onto the emission region.

The laser light 16 from the VCSEL 11 enters the anterior chamber 12 directly and the emergent light from the anterior chamber 12 impinges on the mirror surface 26 of the first optical element 24 directly, from where it is reflected and then coupled back into the anterior chamber 12. It then enters the VCSEL 11 directly from the chamber 12 in order to effect the self-mixing interference.

In this regard, the laser light 16 is preferably not reflected in the anterior chamber 12, but exits the anterior chamber 12 on an opposite side relative to the location of the entry of the laser light 16 into the chamber 12. The beam paths 221, 222 of the laser light 16 and the emergent light 20 are collinear, wherein they extend along a straight axis to which the first optical element 241 is perpendicular. The emission region on the VCSEL 11, the entry point of the laser light 16 into the anterior chamber 12 and the exit point of the emergent light 20 from the anterior chamber 12 lie on a common axis.

In order to allow the laser light 16 or the emergent light 20 to extend along a collinear first and second beam path 221, 222, the anterior chamber 12 is arranged between the first optical element 241 and the VCSEL 11.

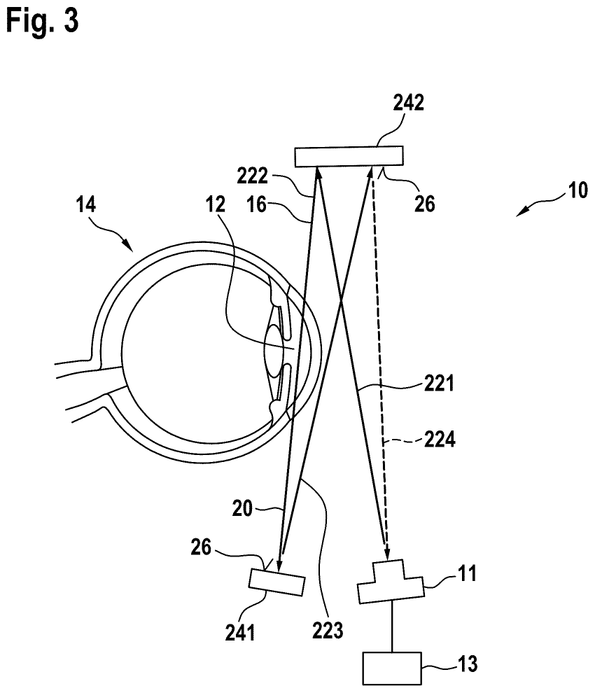

FIG. 3 shows an exemplary embodiment of the device 10 in which an anterior chamber 12 is positioned between the first and a second optical element 241, 242.

The laser light 16 from the VCSEL 11 propagates along a first beam path 221 directly to the second optical element 242 and is reflected at the second optical element 242 at a mirror surface 26, which is oriented such that the second beam path 222 of the laser light 16 passes through the anterior chamber 12. Accordingly, the laser light 16 reflected at the second optical element 242 is coupled into the anterior chamber 12.

Preferably, the coupled laser light 16 passes through the anterior chamber 12 without reflection in the chamber 12 such that the emergent light 20 emerges on an opposite side of the chamber 12. The second beam path 222 passes through the anterior chamber 12 and is reflected at the first optical element 241.

After reflection at the first optical element 241, the emergent light 20 propagates along a third beam path 223 back towards the second optical element 242. In this regard, the emergent light 20 reflected at the first optical element 241 is not coupled into the anterior chamber 12, but is guided past it. For this purpose, the mirror surface 26 of the first optical element 241 is tilted with respect to the second beam path 222 such that the mirror surface 26 is not aligned perpendicular to the second beam path 222.

The first and third beam paths 221, 223 cross one another according to the exemplary embodiment of FIG. 3.

The emergent light is reflected by the second optical element 241 and propagates along a fourth beam path 224 to the VCSEL 11. It is then coupled into the VCSEL 11 in order to effect the self-mixing interference.

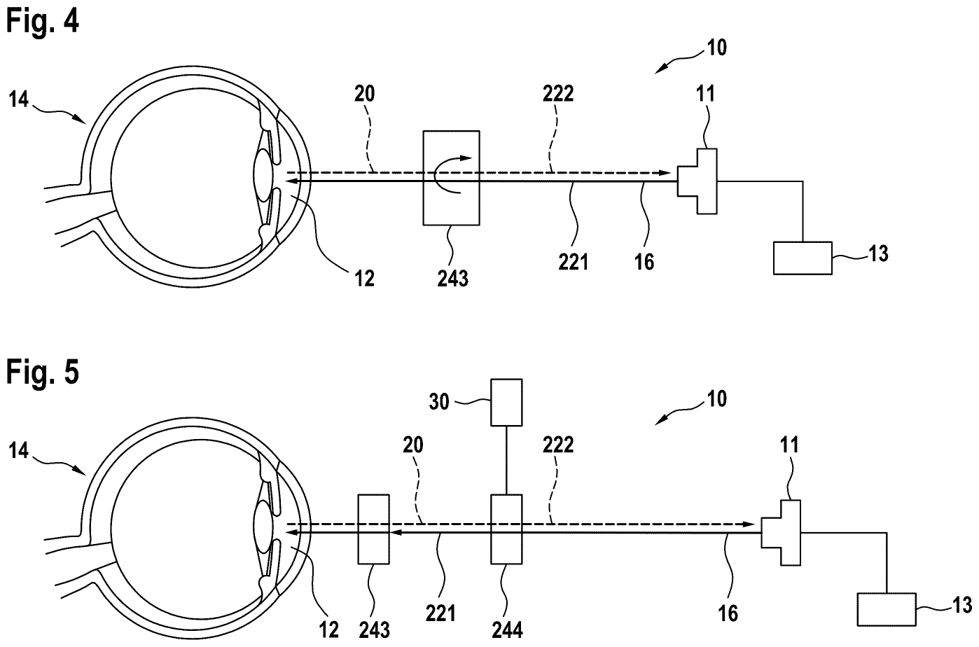

FIG. 4 shows a further exemplary embodiment of the device 10, in which an optical element is provided as a filter for the emergent light 20. The filter represents a third optical element 243 which produces a phase delay of a quarter wavelength in the light passing through it. Here, the third optical element 243 is positioned between the anterior chamber 12 and the VCSEL 11 in a first beam path 221 of the laser light 16 from the VCSEL 11 and a second beam path 222 of the emergent light 20.

The third optical element 243 is a quarter-wave plate. If the laser light 16 from the VCSEL 11 passes through the quarter-wave plate and the linear polarization of the laser light 16 is aligned in the direction of the optical axis of the quarter-wave plate, then the laser light 16 remains linearly polarized.

The emergent light 20, which is rotated by the chiral fluid in the anterior chamber 12 with respect to the laser light 16 from the VCSEL 11 by the angular amount, is also linearly polarized. However, the polarization is also tilted relative to the optical axis of the quarter-wave plate due to the angular amount. If the emergent light 20 now impinges on the quarter-wave plate with the polarization tilted by the angular amount, the emergent light 20 is circularly polarized.

As a result, the polarization can be used to distinguish the emergent light 20 from scattered light or laser light 16 as the laser light 16 from the VCSEL 11 and the emergent light 20 from the anterior chamber 12 exhibit different polarizations.

In FIG. 5, a device 10 is shown which has a fourth optical element 244 which is provided for modulating the laser light 16 or the emergent light 20. The fourth optical element 244 has a liquid crystal module, which is connected to a driver means 30. The liquid crystal module polarizes the laser light 16 before it enters the anterior chamber 12.

The liquid crystal module is controlled by the driver means 30 in such a way that it brings the laser light 16 into at least two different polarization modes or polarization directions. For example, two different linear polarization directions can be set. Alternatively, more than two polarization directions can be set.

For example, the polarization can be adjusted periodically by the driver means 30.

Furthermore, the third optical element 243 can be arranged in the beam path 221, 222 of the laser light 16 or the emergent light 20. For example, the laser light 16 can be tilted once in the direction of the optical axis of the third optical element 243, which is a quarter-wave plate, and once by an angular amount with respect to the optical axis of the third optical element 243. The third optical element 243 can be used to distinguish between the two polarization types or directions as a circular polarization or a modified linear polarization can be produced by tilting the polarization axis with respect to the optical axis of the quarter-wave plate.

In particular, the frequency at which the different polarization modes are set is higher than the frequency of an average human eye movement such that the polarization mode is changed at least once while the eye is at rest. Thus, the glucose concentration in the anterior chamber 12 can be measured using the laser light 16 modulated by the fourth optical element 244, wherein the sensitivity of the laser light 16 can be adjusted to the chiral fluid in the anterior chamber 12.

A liquid crystal module can also be used in any of the further embodiments of FIGS. 1 to 6. The liquid crystal module can be arranged in the beam path of the emergent light 20 or the laser light 16 in any of the embodiments. Furthermore, multiple liquid crystal modules can also be used.

Alternatively or additionally, the light can already be modulated in the VCSEL 11. For example, a piezo element can be attached to the VCSEL 11 that generates a mechanical tension in the VCSEL 11 that has a corresponding orientation with respect to the crystal orientation of the semiconductor material of the VCSEL 11 such that the laser light 16 leaves the VCSEL 11 with a desired polarization. The piezo element can be controlled by the driver means, for example periodically.

Alternatively or additionally, a heating element can be provided which, by heating the semiconductor material, generates mechanical tensions in the semiconductor material that affect the polarization of the laser light 16 as with the piezo element.

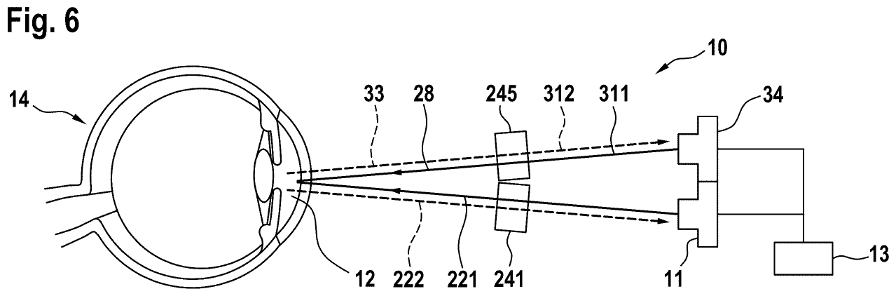

FIG. 6 shows a further exemplary embodiment in which a reference VCSEL 34 is provided in addition to the VCSEL 11, which emits a reference laser light 28 that propagates along a reference beam path 311 to the eye 14.

The polarity of the reference laser light 28 is selected such that it interacts less with the chiral fluid in the anterior chamber 12 than the laser light 16 of the VCSEL 11 of the device 10. Preferably, the reference laser light 28 is completely uniformly circularly polarized when coupled into the anterior chamber 12 as circularly polarized light is not rotated by interaction with the fluid in the eye.

In order for the reference laser light 28 to be circularly polarized, it can pass through a reference optical element 245, which is a quarter-wave plate, wherein the reference laser light 28 from the reference VCSEL 34 is tilted with respect to the optical axis of the quarter-wave plate by an angular amount of, for example, 45°. The circularly polarized reference laser light 28 is reflected as the reference emergent light 33, which is also circularly polarized. The reference emergent light 33 is linearly polarized again by the reference optical element 245. In this regard, it can effect a self-mixing interference in the reference VCSEL 34. This allows conclusions to be drawn with respect to eye movements and/or the glucose concentration in conjunction with the VCSEL 11.

Preferably, the reference optical element 245 and the optical element 243 can be integrated in a single component in an integral manner.

The VCSEL 11 in FIG. 6 is implemented in the device 10 of FIG. 4 by way of example. The laser light 16 of the VCSEL 11 and the reference laser light 28 preferably propagate along nearly collinear beam paths 221, 311. Similarly, the emergent light 20 and the reference emergent light 33 can extend along nearly collinear beam paths 222, 312. For example, a VCSEL array can be used which has at least two emission regions, wherein at least one emission region is provided for the reference laser light 28 and another emission region is provided for the laser light 16.

In another alternative or additional embodiment, the reference laser light 28 of the reference VCSEL 34 can propagate collinearly with the laser light 16 of the VCSEL 11 along a beam path by using an appropriate optical system. The optical system can have an optical fiber, for example.

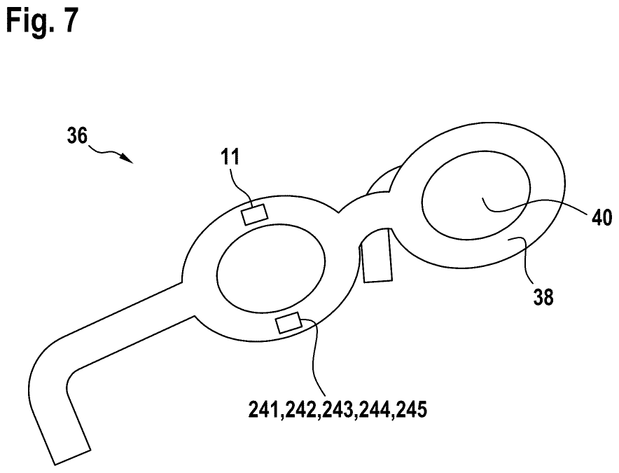

FIG. 7 shows a glasses unit 36 intended to be worn by a user, having at least one of the devices 10 of FIGS. 1 to 6 in the glasses frame 38. In this case, an optical element 241, 242, 243, 244, 245 can be arranged opposite the VCSEL 11 in the glasses frame 38 with respect to a glasses lens 40.

For example, the glasses unit 36 can be a pair of data glasses, virtual reality glasses, augmented reality glasses or conventional eyeglasses. Furthermore, a battery for powering the device and a control system of the device 10 can be attached to the glasses unit 36.

All described exemplary embodiments of the device 10 can determine the glucose concentration at regular time intervals. The glucose measurement is started after a time interval in which no measurement has taken place. The time intervals can be of the same length or individually programmed. For example, the time intervals can be selected such that the glucose measurements are taken at mealtimes, as there is then a high probability that the blood glucose level will rise for technical reasons as a result of food intake.

Blood glucose levels can also be measured during physical activities such as exercise.

At the same time, the control system of the device can indicate to the user what glucose level is present in the chamber 12 and recommend an insulin dose or the intake of glucose accordingly. The recommendation can be displayed on the glasses lens 40, in the eye 14 and/or on a computer such as a smartphone.

A time protocol can be created that shows the history of the glucose concentration in the chamber 12 over time. The protocol is available in digital form and can be read from the device via radio or a physical interface such as USB, for example.

The protocol can be transferred from the device to a computing device such as a smartphone or to a cloud, where it is further processed and made available to a user or a doctor, for example.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SYMBOLS

10 Device
11 VCSEL
12 Chamber
13 Analysis unit
14 Eye
16 Laser light
20 Emergent light
221 First beam path
222 Second beam path
223 Third beam path
224 Fourth beam path
241 First optical element
242 Second optical element
243 Third optical element
244 Fourth optical element
26 Mirror surface
28 Reference laser light
30 Driver means
311 Reference beam path of the reference laser light
312 Reference beam path of the reference emergent light
33 Reference emergent light
34 Reference VCSEL
36 Glasses unit
38 Glasses frame
40 Glasses lens

1. A device for determining a glucose concentration in an anterior chamber of a user's eye, the device comprising:
a VCSEL which emits laser light,
an optical element for influencing the laser light and/or an emergent light, wherein the VCSEL and the optical element are configured such that the laser light enters the anterior chamber of the eye, wherein the emergent light from the anterior chamber penetrates into the VCSEL, and
an analysis unit that analyses a resulting self-mixing interference within the VCSEL to determine the glucose concentration.

2. The device according to claim 1, wherein the optical element has a mirror surface, wherein the mirror surface is arranged in a beam path of the emergent light or the laser light such that the emergent light is reflected on the mirror surface.

3. The device according to claim 1, wherein the anterior chamber is positioned along a beam path between the VCSEL and the optical element.

4. The device according to claim 2, wherein the laser light from the VCSEL enters the anterior chamber directly and the emergent light impinges directly on the mirror surface.

5. The device according to claim 2, wherein the anterior chamber is arranged between the optical element and the VCSEL, wherein the mirror surface is aligned perpendicular to the beam path of the emergent light such that the emergent light is reflected back into the anterior chamber.

6. The device according to claim 1, further comprising a second optical element, wherein the anterior chamber is positioned between the optical element and the second optical element such that the beam path of the laser light passes through the anterior chamber.

7. The device according to claim 6, wherein the beam path of the laser light from the VCSEL passes directly to the second optical element.

8. The device according to claim 6, wherein the beam path of the laser light crosses at least once.

9. The device according to one of claims 6, wherein the beam path of the emergent light extends from the optical element to the second optical element.

10. The device according to claim 1, further comprising a third optical element, which produces a phase delay of a quarter wavelength when the laser light passes therethrough.

11. The device according to claim 10, wherein the third optical element is positioned between the anterior chamber and the VCSEL in a beam path of the laser light from the VCSEL and the emergent light.

12. The device according to claim 1, further comprising a fourth optical element having a liquid crystal module, which influences a polarization direction of the laser light before the laser light enters the anterior chamber, wherein the liquid crystal module is connected to a driver.

13. The device according to claim 1, further comprising a reference VCSEL, which emits a reference laser light that propagates along a reference beam path to the eye.

14. The device according to claim 13, wherein the reference laser light and the laser light propagate collinearly to one another.

15. The device according to claim 1, wherein the optical element is a refractive and/or diffractive lens and/or a lens made of a photonic metamaterial, which is arranged between the VCSEL and the anterior chamber.

16. A glass unit to be worn by a user having a device according to claim 1 such that a user of the glass unit is able to determine his or her own blood glucose level.

17. A method for determining a glucose concentration in an anterior chamber of a user's eye with a device according to claim 1, the method comprising:
emitting the laser light,
causing the laser light to enter or exit into/out of the anterior chamber,
receiving the laser light with the VCSEL,
generating a self-mixing interference within the VCSEL,
analyzing the self-mixing interference with regard to a glucose concentration in the anterior chamber, and
determining the glucose concentration.

18. The method according to claim 17, wherein the determination of the glucose concentration is carried out regularly at specific time intervals such that a chronological protocol of the glucose concentrations at different points in time is created and stored in a memory of the device.

19. The method according to claim 18, wherein the protocol is sent via radio from the device to a computing device.
